(19) United States
(12) Patent Application Publication
(10) Pub. No.: US 2011/0190742 A1
(43) Pub. Date: Aug. 4, 2011

(54) METHOD FOR TREATING KERATOCONUS BY UV RADIATION AND A DEVICE FOR CARRYING OUT SAID METHOD (VARIANTS)

(76) Inventor: Sergey Igorevich Anisimov, Moscow (RU)

(21) Appl. No.: 13/059,024

(22) PCT Filed: Mar. 12, 2009
(86) PCT No.: PCT/RU2009/000121

§ 371 Date: Mar. 7, 2011

(30) Foreign Application Priority Data
Aug. 14, 2008 (RU) 2008133298

(51) Int. Cl. A61B 18/18

(52) U.S. Cl. 606/5

(57) ABSTRACT

The invention relates to ophthalmology and is used for treating keratoconus. The inventive method comprises forming one or more radiation areas of different shape, comprising concentric circles, arcs, parallel lines, cells, grids, spirals or other geometrical figures. In the other variant, the method comprises polarizing UV radiation and adjusting the depth of the UV radiation action by directing the UV radiation polarization plane at an angle of 1-180° to the plane of light polarization by the cornea, thereby using the effect of light polarization by the cornea. The device for treating keratoconus comprises, positioned on the optical axis common with an UV radiation source optical focusing elements and a diaphragm. The diaphragm is situated on the optical axis common with the UV radiation source and is made in the form of a mask with alternating transparent and shaded elements in the form of concentric circles, arcs, parallel lines, cells, grids, spirals or other geometrical figures. In the other variant, the device comprises, situated on the optical axis common with the UV radiation source, the optical focusing elements, the diaphragm and a polarisor.

### (i) SCOPE OF USE

The invention relates to medicine, more specifically to ophthalmology and can be used for treating keratoconus by UV radiation.

### (ii) PRIOR KNOWLEDGE

It is already known a method and a device for treating keratoconus by UV radiation that affects the cornea with full aperture (Wollensak G., Spörl E., Seiler T. Treatment of keratoconus by collagen cross linking. Der Ophthalmologie: Zeitschrift der Deutschen Ophthalmologischen Gesellschaft, 2003, January, 100(1): p. 44-49).

However, this method is traumatic because of the adverse effects of UV radiation on the cornea.

The influence of UV radiation on the cornea results collagen molecules photopolymerization. Photopolymerization is activated by non-toxic water-soluble photomediators, and their radiation absorption at depth should protect the deep tissue of the cornea, in particular, endothelial cells against damage. Such photomediator may be riboflavin, in particular, its 1% solution. The process of collagen ligation is called cross-linking and can occur at different levels of a complex collagen structure. In case of cross-linking there are additional cross-links in tropocollagen that form the primary structure of this protein, additional cross-linking between the primary collagen structures may also appear, and, finally new bridges between the collagen microfibrils may be formed. All this leads to increased cornea collagen aggregation, its thickening and hardening. The clinical effect is caused by these changes of corneal biomechanical parameters that allow stabilizing the corneal curvature in keratoconus.

However, irradiation of the cornea by full aperture UV source, providing uniform illumination of the entire cornea area, can cause photo-burn, and the imperfect cornea thickness, smaller than the depth of UV radiation exposure, can affect damage of endothelial cells with the progression of complications such as corneal dystrophy. In some cases this requires the use of special mixtures of riboflavin with dextran that provide cornea thickening due to temporary edema. This respectively reduces the effect of the collagen cornea cross-linking in case of keratoconus.

### (iii) DISCLOSURE OF THE INVENTION

The technical result achieved when working of the invention is to reduce the adverse effects of exposure.

Mentioned technical result is achieved by a method of treating keratoconus implemented as follows. Local anesthesia is performed. Photomediator is instilled in the eye, in particular, a 1% solution of riboflavin for 30 minutes every 2 minutes by one drop. Cornea is irradiated with UV with a power not exceeding 5 mW/cm² from 1 to 90 minutes, forming one or more radiation areas of different shape, including concentric circles, grids, cells, spirals, arcs, parallel lines or other geometric figures. This is to form a kind of reinforcement, necessary cornea strength is achieved with a smaller impact area, thus preserving the efficacy of treatment and reduces risk of side effects or weakens them, ensures the formation of one or more cornea exposure areas in the form of concentric circles, grids, cells, spirals, arcs, parallel lines or other geometric figures.

Mentioned technical result is also achieved by the fact that the method of treatment is implemented as follows. Performed local anesthesia. Local anesthesia is performed. Photomediator is instilled in the eye, for example, a 1% for 30 minutes every 2 minutes by one drop. UV radiation is polarized, the polarization plane with respect to the cornea polarization plane is set at an angle of 1 to 180 degrees. Cornea is irradiated by UV radiation with a power not exceeding 5 mW/cm² from 1 to 90 minutes. The UV radiation polarization and the direction of its polarization plane with respect to the cornea polarization plane at an angle of 1 to 180 degrees makes it possible, due to the polarization effect of light polarization of the cornea, adjusting the depth of UV radiation, reducing UV radiation exposure on endothelial cells sensitive to photo-damage that, in turn, maintaining the treatment effectiveness, reduces the risk of any cornea UV radiation adverse effects severity level.

Technical result is also achieved because the device for the treatment of keratoconus comprises a body with a UV radiation source located on it, connected with power supply and control, optical focusing elements located on the same optical axis with a source of UV radiation, and the diaphragm.

Diaphragm is designed as a mask of alternating transparent and shaded areas with a shape of concentric circles, grids, cells, arcs, parallel lines or other geometric figures. Aperture is designed to be located on the same optical axis with a UV radiation source and the optical focusing elements.

Diaphragm can be installed in the device body frame or brought out its limits.

In another modification, the technical result is achieved by the device for the keratoconus treatment comprises a body frame with and installed UV radiation source, connected with it power supply and control, optical focusing elements located on the same optical axis with UV radiation source, the diaphragm and a polarizer, designed for installation on the same optical axis with UV radiation source and optical focusing elements, made with the possibility of setting UV polarization plane against the cornea polarization plane the within 1-180 degrees.

Polarizer can be installed in the device or brought out its limits.

### (iv) EXAMPLES OF WORKING OF THE INVENTION

Method of treating and the device is illustrated in the figures.

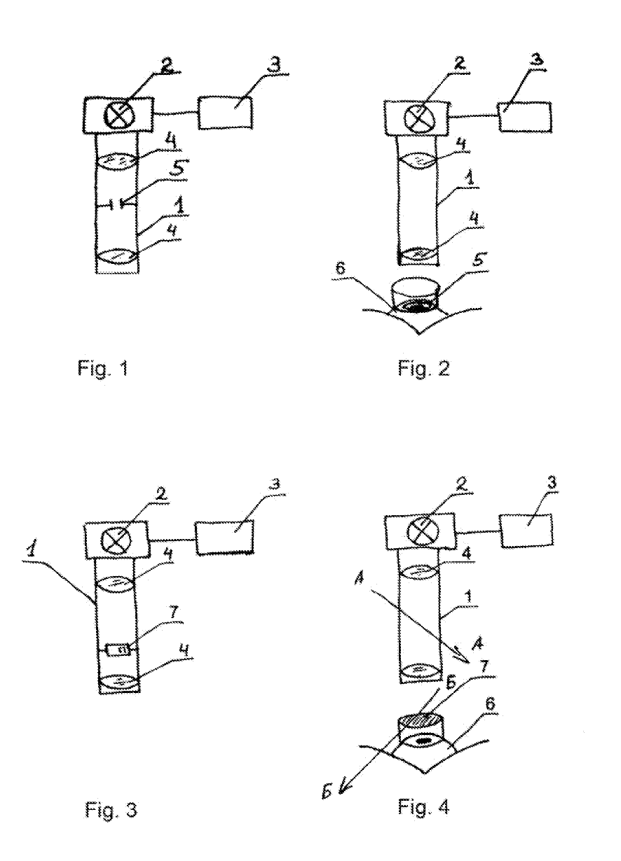

FIG. 1 illustrates a block diagram of the device with a diaphragm in the body frame.

FIG. 2 illustrates a block diagram of the device with a diaphragm outside the body frame.

FIG. 3 illustrates a block diagram of the device with a polarizer in the body frame.

FIG. 4 illustrates a block diagram of the device with a polarizer outside the body frame.

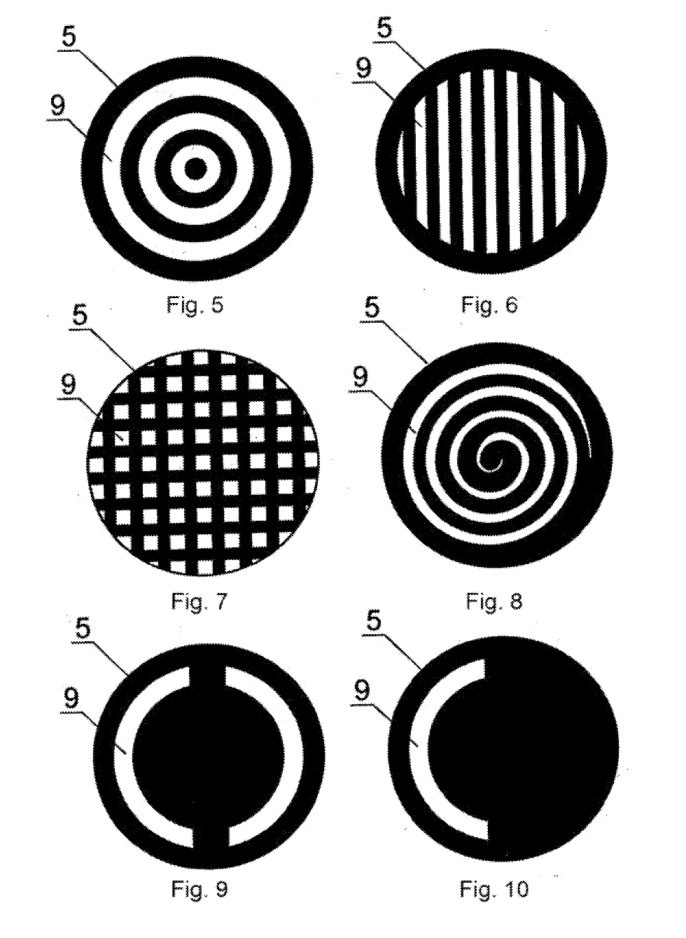

FIG. 5-10— kind of the diaphragm in isometric view with a mask in the form of various geometric figures.

Device for the treatment of keratoconus comprises a body frame 1 where the UV radiation source 2 with a wavelength of 350-380 nm and the elements of the optical system 4 of quartz glass are located. UV radiation source is connected to the power supply and control unit 3, which provides electricity supply, the regulation time and the power of the device. The device also contains a diaphragm 5, made in the form of a mask of alternating transparent and shaded areas with the shape of concentric circles, grids, cells, arcs, parallel lines or other geometric figures, which is installed on the same optical axis with the elements of the optical system 4 and UV radiation source. In this case, the diaphragm 5 can be placed in the body frame or outside it, and when the device is placed on one optical axis with UV radiation source. Treatment is carried out as follows.

After preliminary local anesthesia photomediator is instilled in the eye, in particular, 1% solution of riboflavin is instilled within 30 minutes and is irradiated with UV focused through an aperture within 1-90 minutes. Due to the corneal collagen cross-linking chemical reaction in the corneal appear areas of increased strength. Diaphragm is designed as a mask of alternating transparent and shaded areas, with the shape of concentric circles, grids, cells, arcs, parallel lines or other geometric figures (FIG. 5-10) that allows to create the irradiation area in the cornea, forming a kind of reinforcement, thus achieving the required cornea strength with a smaller impact area, which in turn, maintaining the treatment effectiveness, reduces the risk of weakening or possible side effects of the procedure.

Another type of the device comprises the body frame 1 where the UV radiation source 2 with wavelength 350-380 nm and the elements of the optical system 4 of quartz glass are located. UV radiation source connected to the power supply and control unit 3, which supply electricity, provide time and power regulation of UV radiation source. The device also contains a diaphragm 5, which is installed on the same optical axis with the elements of the optical system 4 and a UV radiation source of. In addition, the device comprises a polarizer 7, located on the optical axis of the body frame or outside the body frame. Polarization plane in polarizer is rotatable within 360 degrees and can polarize ultraviolet radiation. Through the use of the effect of light polarization by the cornea, directing the UV radiation polarization plane in relation to the cornea polarization plane at an angle between 1 and 180 degrees it is possible to regulate the UV radiation depth, reducing UV radiation exposure on photo-damage sensitive endothelial cells, which in turn, maintaining the effectiveness of treatment, reduces the risk of weakening or possible side effects of the procedure. FIG. 4 shows one of the positions of the polarizer axis A-A—relating to the B-B—corneal polarization axis. Treatment is carried out as follows.

With the polarimeter is defined cornea polarization plane. After preliminary local anesthesia for 30 minutes photomediator is instilled in the eye, for example, 1% solution of riboflavin. On the same optical axis with the UV radiation source polarizer is set, the polarization plane of which is rotated relative to the light polarization of the cornea plane at an angle of 1 to 180 degrees and the cornea is irradiated through a polarizer by a focused UV light for 1-90 minutes. Due to the corneal collagen cross-linking chemical reaction areas of the increased strength appear. The described polarizer installation helps to adjust the depth of UV radiation exposure, reducing UV radiation exposure on photo-damage sensitive endothelial cells, which in turn, maintaining the effectiveness of treatment, reduces the risk of weakening or possible side effects of the procedure.

Below are listed examples of specific performance of the method.

### EXAMPLE 1

### Patient A., Aged 26

Diagnosis: keratoconus of both eyes. Visual acuity before surgery:
right eye—0,01 sph—4,0 D, cyl—4,0 D ax 85°
left eye—0,2 sph—1,5 D, cyl—1,5 D ax 95°

Intensive disease progression is present over the past six months.

Surgery was carried out according to the suggested method. After the local anesthesia photomediator instillation is provided, namely the solution of 1% riboflavin within 30 minutes. Then on the corneal surface the diaphragm image of concentric rings formed by the UV source was focused. The exposition was 1 minute.

The next day after surgery—right eye calm, zero degree reaction, no soreness, and no signs of cornea damage seen on slit lamp examination. Control examination after 1 year showed no disease progression in the right eye. The same method was used for the treatment of the left eye.

### EXAMPLE 2

### Patient B., 28 Years Old

Diagnosis: keratoconus of both eyes. Visual acuity before surgery:
right eye —0,01 sph—2,0 D, cyl—5,0 D ax 65°
left eye—0,1 sph—1,5 D, cyl—3,5 D ax 105°

Intensive disease progression is present over the past six months.

Surgery was carried out according to the suggested method. After the local anesthesia photomediator instillation is provided, namely the solution of 1% riboflavin within 30 minutes, on the surface of the cornea aperture in the form of parallel lines was set, it was formed by the UV source, the direction of the lines was chosen to be parallel to the astigmatism axis, i.e., parallel to the meridian 65 degrees. Diaphragm was fixed with a vacuum ring. The exposition was 30 minutes.

The next day after surgery—right eye calm, zero degree reaction, no soreness, and no signs of cornea damage seen on slit lamp examination. Control examination after 1 year showed no disease progression in the right eye. The same method was used for the treatment of the left eye.

### EXAMPLE 3

### Patient Sh., Aged 38

Diagnosis: keratoconus of both eyes. Visual acuity before surgery:
right eye—0,01 sph—0,5 D, cyl—4,0 D ax 75°
left eye—0,2 cyl—3,75 D ax 80°

Deterioration of vision during the last year.

Surgery was carried out according to the suggested method. Cornea polarization plane was defined with the polarimeter. Local anesthesia was applied. After photomediator instillation, namely the solution of 1% riboflavin within 30 minutes, the polarization plane of the device polarizer deployed at 90 degrees to the cornea polarization plane. On the right eye cornea surface was focused spot formed by the UV source. The exposition was 40 minutes.

The next day after surgery—right eye calm, zero degree reaction, no soreness, and no signs of cornea damage seen on slit lamp examination. Control examination after 1 year showed no disease progression in the right eye.

### EXAMPLE 4

### Patient A., Aged 24

Diagnosis: bilateral keratoconus. visual acuity before surgery:
right eye—0,05 sph—0,25 D, cyl—4,5 D ax 65°
left eye—0,2 cyl—3, 5 D ax 105 °

Deterioration of vision during the last year.

Surgery was carried out according to the suggested method. Cornea polarization plane was defined with the polarimeter. Local anesthesia was applied. After photomediator instillation, namely the solution of 1% riboflavin within 30 minutes, the polarization plane of the device polarizer deployed at 90 degrees to the cornea polarization plane. On the right eye cornea surface was focused spot formed by the UV source. The exposition was 90 minutes.

The next day after surgery—right eye calm, zero degree reaction, no soreness, and no signs of cornea damage seen on slit lamp examination. Control examination after 1 year showed no disease progression in the right eye.

1. Method of treating keratoconus by UV radiation, which comprises applying local anesthesia, photomediator instillation, UV radiation on the cornea for 1-90 minutes, wherein one or more UV radiation exposure areas of different shapes are formed, including concentric circles, arcs, parallel lines, cells, bars, spirals or other geometric figures.

2. Method of treating keratoconus by UV radiation, which comprises applying local anesthesia, photomediator instillation, UV radiation on the cornea for 1-90 minutes, wherein UV radiation is polarized, polarization plane with respect to the cornea polarization plane is located at an angle of 1 to 180 degrees.

3. Device for treating keratoconus by UV radiation, which comprises a body frame with UV source located inside, connected power supply and control unit, located on the same optical axis with a UV source, focusing optical elements and diaphragm, wherein the diaphragm is designed as a mask with alternating transparent and shaded areas having the shape of concentric circles, grids, cells, arcs, parallel lines or other geometric figures, the diaphragm is designed to be located on the same optical axis with UV source.

4. The device of claim 3, wherein the diaphragm is installed inside the device.

5. The device of claim 3, wherein the diaphragm is outside the body frame and is located on the cornea, or between the device and the cornea.

6. Device for treating keratoconus by UV radiation, which comprises a body frame with UV source located inside, connected power supply and control unit, located on the same optical axis with a source of UV radiation of the optical focusing element and the diaphragm, wherein it contains a polarizer designed for placement on the same optical axis with UV source and performed with the possibility of setting UV polarization plane against the cornea polarization plane in the range from 1 to 180 degrees.

7. The device of claim 6, wherein the polarizer is installed in the device.

8. The device of claim 6, wherein the polarizer is outside the body frame and is located on the cornea, or between the device and the cornea.
